(19) United States
(12) Patent Application Publication
(10) Pub. No.: US 2025/0006339 A1
(43) Pub. Date: Jan. 2, 2025

(54) SYSTEM AND METHOD FOR REAL-TIME FITNESS TRACKING AND SCHEDULING

(71) Applicant: Timothy Coyle, New York, NY (US)

(72) Inventor: Timothy Coyle, New York, NY (US)

(21) Appl. No.: 18/884,242

(22) Filed: Sep. 13, 2024

Related U.S. Application Data
(63) Continuation-in-part of application No. 17/154,349, filed on Jan. 21, 2021.
(60) Provisional application No. 62/964,172, filed on Jan. 22, 2020.

(51) Int. Cl. G16H 20/30; G16H 10/60

(52) U.S. Cl. CPC G16H 20/30; G16H 10/60

(57) ABSTRACT

A computer system configured to implement a method for real-time fitness tracking and scheduling is described herein. The computer system receives health data, a first health goal for completion during a first time period, and a second health goal for completion during a second time period for a user. A health profile is generated that includes the health data, the first health goal, and the second health goal. The health data is implemented in if-then scenarios to determine a first wellness action for the user to complete during a first time period to achieve the first health goal and a second wellness action for the user to complete during a second time period to achieve the second health goal. A health and wellness program is created for the user based on the first wellness action and the second wellness action.

### CROSS-REFERENCE TO RELATED APPLICATIONS

This application is a U.S. Continuation-In-Part Utility Application entitled, “SYSTEM AND METHOD FOR REAL-TIME FITNESS TRACKING AND SCHEDULING” which claims priority to co-pending U.S. Non-Provisional patent application Ser. No. 17/154,349 filed Jan. 21, 2021 entitled, “SYSTEM AND METHOD FOR REAL-TIME FITNESS TRACKING AND SCHEDULING” that claims priority to U.S. Provisional Patent Application No. 62/964,172, filed on Jan. 22, 2020 entitled, “SYSTEM AND METHOD FOR REAL-TIME FITNESS TRACKING AND SCHEDULING” the contents of which are hereby fully incorporated by reference.

### FIELD OF THE EMBODIMENTS

The field of the invention and its embodiments relate to a method for real-time fitness tracking and scheduling. In particular, the present invention and its embodiments provide a method for real-time fitness tracking and scheduling that incorporates, via an algorithm, health data of a user in if-then scenarios to determine user-specific wellness actions to complete to achieve health goals and adapt to provide new goals, new suggestions, and new guidance over time. Moreover, the present invention and its embodiments provide a method for real-time fitness tracking and scheduling that converts to unique actionable recommendations that evolve and adapt to the user's input over time.

### BACKGROUND OF THE EMBODIMENTS

A person's health is a combination of multiple factors, including medical data (such as a known health problem of the user, a health problem of a family member associated with the user, a physiological or biochemical measurement of the user, etc.), genetic data (such as genomic information), nutritional data (such as types of foods eaten by the user, a number of daily calories consumed by the user, a quantity of meals consumed daily by the user, etc.), fitness data (such as a type of exercise routine engaged in by the user, a type of workout engaged in by the user, a length of time spent on the exercise routine, a number of calories burned during the workout, etc.), and environmental data (such as lifestyle choices of a user). Health and fitness professionals typically assess only one or two of these factors when creating a health and wellness program for an individual to achieve a health goal. For example, a doctor may only assess medical data associated with a user, whereas a physical trainer may only assess fitness data associated with the user when creating a health and wellness program for the user. Thus, current solutions fail to adequately assess the relationships between these factors and also fail to generate or provide a complete health and wellness program for an individual to achieve one or more health goals. Thus, a need exists for a method for real-time fitness tracking and scheduling that incorporates, via an algorithm, health data of a user in if-then scenarios to determine user-specific wellness actions to complete to achieve health goals.

### Review of Related Technology:

U.S. Pat. No. 8,690,578 pertains to an illustrative mobile computing device executing weight, nutrition, health, behavior and exercise application software serving as a simulated combination personal trainer and dietician/nutritionist for the user using comprehensive databases storing personalized health, nutrition and exercise information. A mobile computing device, such as a smartphone, executing such software monitors, tracks and/or adjusts caloric intake, energy expenditure considering nutritional information and behavioral factors. The mobile computing device receives food consumption, exercise-related, behavior and other input using speech input and the device's GPS subsystem to ease data entry burden on users and to promote continued long-term usage. The system rewards user goal achievement in an automatic, seamless manner, though, for example, downloading music, books, or other media. In illustrative implementations, the system assists users to make healthy food and exercise choices by using a comprehensive color code system to identify good choices, bad choices and those in between.

U.S. Published Patent Application No. 2012/0094258 pertains to a system and method for automated personalized and community-specific eating and activity planning are provided that are linked to tracking with automated multimodal item identification and size estimation and enable and integrate health and other user data streams and enables rewards and links to healthy eating and activity partners based on that data. The system and method also provide personalized wellness recommendations. The system and method also enables action, such as single click ordering of the healthy meals or shopping list on one's plan from local restaurants and grocery stores, and receipt of mobile vouchers and coupons with a unique validation system for use at retailers.

U.S. Published Patent Application No. 2014/0156308 pertains to a system and method provide health-related information. A user interface on a computing device may provide assessment information associated with an assessment of a user's health. Further, sensed information associated with at least one of biological information, physiological information and physical activity of the user can be received from a different device which is configured to sense information. Moreover, a processing subsystem that includes a processor and processor readable media can be configured to process the sensed information, via, to provide processed user information, and to determine health-related information, via the processing subsystem, using the assessment information and the processed user information. Furthermore, the processed user information and the health-related information can be transmitted via a communication subsystem to the computing device, and the health-related information can be provided at the computing device via the user interface substantially contemporaneously with the reception of the sensed information.

Various methods for real-time fitness tracking and scheduling are known in the art. However, their means of operation are substantially different from the present disclosure, as the other inventions fail to solve all the problems taught by the present disclosure. The present invention and its embodiments provide a method for real-time fitness tracking and scheduling. In particular, the present invention and its embodiments provide a method for real-time fitness tracking and scheduling that that incorporates, via an algorithm, health data of a user in if-then scenarios to determine user-specific wellness actions to complete to achieve health goals.

### SUMMARY OF THE EMBODIMENTS

The present invention and its embodiments provide a method for real-time fitness tracking and scheduling. In particular, the present invention and its embodiments provide a method for real-time fitness tracking and scheduling that incorporates, via an algorithm, health data of a user in if-then scenarios to determine user-specific wellness actions to complete to achieve health goals and adapt to provide new goals, new suggestions, and new guidance over time. Moreover, the present invention and its embodiments provide a method for real-time fitness tracking and scheduling that converts to unique actionable recommendations that evolve and adapt to the user's input over time.

A first embodiment of the instant invention describes a method executed by a health engine of a computing device for real-time fitness tracking and scheduling. The method includes receiving health data pertaining to a user and receiving a first health goal of the user for completion during a first time period and a second health goal of the user for completion during a second time period. In some implementations, the health goals may be on different, but parallel timelines from the same or similar sources. In some examples, the health data pertaining to the user may be received from one or more wireless health devices tracking one or more biometric parameters of the user. In other examples, the health data, the first health goal, and/or the second health goal of the user may be received from user input in response to a health questionnaire.

The health data may include medical data, genetic data, nutritional data, fitness data, subjective measures (e.g., self-reported by the user), and/or environmental data, among other data not explicitly listed herein. In some examples, the health data may additionally include stories from the user that include the medical data, the genetic data, the nutritional data, the fitness data, and/or the environmental data. The medical data may include: a known health problem of the user, a prior health problem of the user, a current health problem of the user, a health problem of a family member associated with the user, and/or a physiological or biochemical measurement of the user, among other data not explicitly listed herein. Examples of the physiological or biochemical measurement of the user may include: a heart rate measurement, a resting metabolic rate (RMR) measurement, an oxygen consumption (VO2) level measurement, a weight measurement, a body fat measurement, a visceral fat measurement, a muscle mass measurement, a measurement of body water of the user, a body mass index (BMI) measurement, a bone mass measurement, and/or a blood glucose level measurement, among other information not explicitly listed herein.

Genetic data may include genomic information data. Moreover, examples of the nutritional data may include information, such as: types of foods eaten by the user, a number of daily calories consumed by the user, a quantity of meals consumed daily by the user, a quantity of snacks consumed daily by the user, a type of snacks consumed daily by the user, a type of beverage consumed daily by the user, and/or a quantity of beverages consumed daily by the user, among other information not explicitly listed herein. Examples of the fitness data may include: a type of exercise routine engaged in by the user, a type of workout engaged in by the user, a length of time spent on the exercise routine, a length of time spent on the workout a number of calories burned during the exercise routine, a number of calories burned during the workout, a heart rate achieved during the exercise routine, and/or a heart rate achieved during the workout, among other information not explicitly listed herein. Examples of the environmental data may include a lifestyle choice of the user, such as: a sleep habit of the user, a stress level of the user, a type of learner the user is (e.g., a visual learner, an auditory learner, or a heuristic learner), a smoking habit of the user, and/or an alcohol intake habit of the user, among other information not explicitly listed herein.

Then, the method generates a health profile of the user that includes the health data, the first health goal, and the second health goal. The health data is then implemented or incorporated into if-then scenarios to determine a first wellness action for the user to complete during a first time period to achieve the first health goal and a second wellness action for the user to complete during a second time period to achieve the second health goal. The first time period may be a current time period and the second time period may be a future time period, in examples. In other examples, the first time period may be a future time period and the second time period may be a current time period. A health and wellness program is then generated or created in the health profile for the user based on the first wellness action and the second wellness action.

A second embodiment of the instant invention describes a computer system. The computer system comprises one or more processors, one or more memories, and one or more computer-readable hardware storage devices. The one or more computer-readable hardware storage devices contain program code executable by the one or more processors via the one or more memories to implement a method for real-time fitness tracking and scheduling. According to the method, health data pertaining to the user is first received. The health data may include: medical data, subjective measures (e.g., self-reported by the user), genetic data, nutritional data, fitness data, and/or environmental data, among other data not explicitly listed herein. Then, a first health goal of the user for completion during a first time period and a second health goal of the user for completion during a second time period is received. Next, a health profile for the user is generated, which includes the health data, the first health goal, and the second health goal.

Then, the health data is implemented or incorporated, via the algorithm, in if-then scenarios to determine a first wellness action for the user to complete during a first time period to achieve the first health goal and a second wellness action for the user to complete during a second time period to achieve the second health goal. In some examples, the first time period is a current time period and the second time period is a future time period. In other examples, the first time period is a future time period and the second time period is a current time period. A health and wellness program is then generated or created in the health profile for the user based on the first wellness action and the second wellness action.

According to examples, the method may further include receiving user activity information pertaining to the user's participation in the health and wellness program and/or tracking metrics of the user participation in the health and wellness program. In response to this information, the health profile may be updated based on the user's participation in the health and wellness program. Then, one or more notifications, messages, and/or alerts may be generated and displayed to the user based on the user's participation. The one or more notifications, messages, and/or alerts may include recommendations on how the user can increase performance in the health and wellness program.

A third embodiment of the instant invention includes a method for real-time fitness tracking and scheduling. According to the method, health data pertaining to a user is first received, which includes: medical data, genetic data, subjective measures (e.g., self-reported by the user), nutritional data, fitness data, and/or environmental data. In some examples, the health data may be received from one or more wireless health devices tracking one or more biometric parameters of the user and/or from user input in response to a health questionnaire. Then, a first health goal of the user for completion during a first time period and a second health goal of the user for completion during a second time period is received. In some examples, the first time period is a current time period and the second time period is a future time period. In other examples, the first time period is a future time period and the second time period is a current time period.

Next, a health profile of a user is generated, which includes: the health data, the first health goal, and/or the second health goal. Then, the health data is implemented or incorporated, via an algorithm, in if-then scenarios to determine a first wellness action for the user to complete during a first time period to achieve the first health goal and a second wellness action for the user to complete during a second time period to achieve the second health goal. Next, a health and wellness program is created in the health profile for the user based on the first wellness action and the second wellness action.

In some examples, the method further includes assessing user participation in the health and wellness program from received user activity information and tracked metrics. Then, the health profile is updated based on the user's participation in the health and wellness program. One or more notifications, messages, and/or alerts are then generated and displayed to the user based on the user's participation. The one or more notifications, messages, and/or alerts may include recommendations on how the user can increase performance in the health and wellness program. In some examples, the one or more notifications, messages, and/or alerts are generated by a healthcare provider supervising the user's participation in the health and wellness program. In additional examples, the user may receive points and/or rewards based on a level of user's participation in the health and wellness program to encourage participation in the health and wellness program.

In general, the present invention succeeds in conferring the following benefits and objectives.

It is an object of the present invention to provide a method for real-time fitness tracking and scheduling.

It is an object of the present invention to provide a method for real-time fitness tracking and scheduling based on processes found in scientific literature.

It is an object of the present invention to provide both a macro-analysis and a micro-analysis of health data of a user to provide an encompassing real-time fitness tracking and scheduling method.

It is an object of the present invention to provide both a macro-analysis and a micro-analysis of health data of a user to provide long-term fitness and/or health management.

It is an object of the present invention to provide both a macro-analysis and a micro-analysis of health data of a user to provide long-term fitness and/or health wellness.

It is an object of the present invention to identify user health priorities and establish a user-directed fitness pacing system that is both flexible and responsive to the user.

It is an object of the present invention to assess medical data, genetic data, nutritional data, fitness data, and/or environmental data of a user, implement the health data in if-then scenarios, and generate wellness actions, based on this data and these scenarios, for the user to complete to meet user-specific health goals.

It is an object of the present invention to assess medical data, genetic data, nutritional data, fitness data, and/or environmental data of a user, implement, via an algorithm, the health data in if-then scenarios, and generate wellness actions, based on this data and these scenarios, for the user to complete to meet user-specific health goals.

It is an object of the present invention to generate one or more notifications, messages, and/or alerts to a computing device for display to the user to provide recommendations on how the user can increase his/her performance in user-specific a health and wellness program to meet user-specific health goals.

### BRIEF DESCRIPTION OF THE DRAWINGS

The present disclosure may be better understood, and its numerous features and advantages made apparent to those skilled in the art by referencing the accompanying drawings. The use of the same reference symbols in different drawings indicates similar or identical items.

FIG. 1-FIG. 2 depict perspective views of a computer system configured to implement a method for real-time fitness tracking and scheduling, according to at least some embodiments described herein.

FIG. 3 depicts a graphical representation of an algorithm configured to implement a method for real-time fitness tracking and scheduling, according to at least some embodiments described herein.

FIG. 4 depicts a perspective view of an intake screen of a health engine associated with a computer system configured to implement a method for real-time long-term and short-term fitness and/or health/wellness tracking and scheduling, according to at least some embodiments described herein.

FIG. 5-FIG. 6 depict perspective views of a medical history screen of a health engine, according to at least some embodiments described herein.

FIG. 7 depicts a perspective view of pain points associated with a medical history screen of a health engine, according to at least some embodiments described herein.

FIG. 8 depicts a perspective view of a login screen associated with a health engine, according to at least some embodiments described herein.

FIG. 9-FIG. 10 depict perspective views of a health profile associated with a health engine, according to at least some embodiments described herein.

FIG. 11-FIG. 13 depict perspective views of an equipment screen associated with a health engine, according to at least some embodiments described herein.

FIG. 14 depicts a perspective view of a health profile associated with a health engine, where the health profile depicts a workout session, according to at least some embodiments described herein.

FIG. 15-FIG. 16 depict perspective views of a representation of an individual exercise associated with a workout session of a health profile, according to at least some embodiments described herein.

FIG. 17 depicts a perspective view of a nutrition module associated with a health profile, according to at least some embodiments described herein.

FIG. 18-FIG. 19 depict perspective views of individual exercises of a workout session associated with a health profile, according to at least some embodiments described herein.

FIG. 20-FIG. 21 depict perspective views of an alert displayed via a health profile, according to at least some embodiments described herein.

FIG. 22 is a block diagram of a computing device included within the computer system of FIG. 1 and/or FIG. 2 that is configured for real-time fitness tracking and scheduling, in accordance with embodiments of the present invention.

FIG. 23 is a diagram of an example process for the real-time fitness tracking and scheduling application implementing the health engine, in accordance with embodiments of the present invention.

FIG. 24 is a flowchart illustrating a method, according to some embodiments of the present disclosure.

FIG. 25 is a flowchart further illustrating the method from FIG. 24, according to some embodiments of the present disclosure.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Described herein is a method and system to create a user-specific and personalized health and wellness program. The instant invention provides individualized steps that create a truly personalized, stepwise approach or program that unfolds/evolves as the user participates over time.

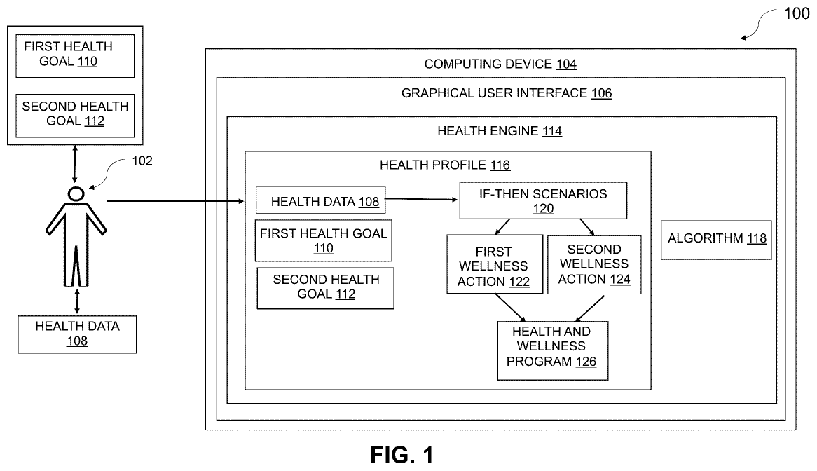

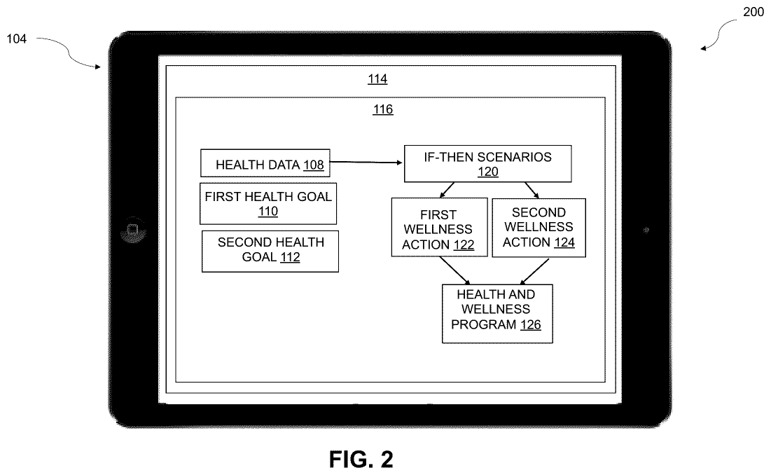

FIG. 1 and FIG. 2 depict a computer system 100 and a computer system 200, respectively, configured to implement a method for real-time fitness tracking and scheduling. Specifically, the computer system 100 and the computer system 200, respectively, implement the method for the real-time fitness tracking and scheduling, which includes providing both a macro-analysis and a micro-analysis of health data of a user, assessing the health data in if-then scenarios to determine user-specific wellness actions for a user to complete to achieve user-specific health goals, and generating a health and wellness program based on these user-specific wellness actions to achieve the user-specific health goals. Specifically, the micro-analysis includes decisions that guide the macro-analysis based on the user-specific health goals and needs. Moreover, the method for the real-time fitness tracking and scheduling identifies user health priorities and establishes a user-directed fitness pacing system that is both flexible and responsive to a user 102. The user-directed fitness pacing system provides long-term fitness management for the user 102.

The computer system 100 may include a computing device 104. The computing device 104 may be a computer, a laptop computer, a smartphone, and/or a tablet, among other examples not explicitly listed herein. The computing device 104 may comprise a health engine 114 that may execute the method for real-time fitness tracking and scheduling. In other examples, the health engine 114 may be a health application, a health software program, a health service, or a health software platform configured to be executable on the computing device 104. The user 102 may interact directly with the health engine 114 via a graphical user interface (GUI) 106 of the computing device 104.

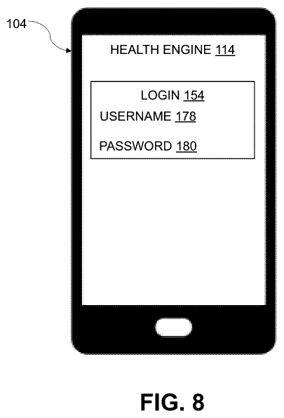

As shown in FIG. 8, the health engine 114 may receive, from the user 102, login credentials such that the user 102 may login 154 to and interact with the health engine 114. The login credentials may include a username 178, a password 180, a biometric identification means (e.g., fingerprint identification, face recognition identification, palm print identification, iris recognition, retina recognition, etc.), etc. In response, the health engine 114 identifies the user 102 based on the login credentials.

Identification of the user 102 may include information such as: a name of the user 102, a telephone number of the user 102, an address of the user 102, etc. In some examples, identifying the user 102 based on the login credentials may include determining that the user 102 has a health profile 116 associated with the health engine 114. If it is determined that the user 102 does not yet have the health profile 116, the health engine 114 may prompt the user 102 to create such profile. Responsive to creation of such profile, the health engine 114 may grant the user 102 access to the health engine 114.

Further, the health engine 114 may receive health data 108 pertaining to the user 102. The health data 108 may be received by the health engine 114 from one or more wired or wireless health devices tracking one or more biometric parameters of the user 102. The one or more wired or wireless health devices may continuously track and update the one or more biometric parameters of the user 102. Examples of these wired or wireless health devices include: watches, bracelets, wristbands, ankle bands, rings, or necklaces, among other examples not explicitly listed herein. Examples of the one or more biometric parameters of the user 102 include: a heartrate of the user 160, a quantity of calories burned by the user 160, and/or a blood pressure of the user 160, among other parameters not explicitly listed herein.

In other examples, the health data 108 pertaining to the user 102 may be received from user input (e.g., audio or textual) via the GUI 106 in response to the health questionnaire (e.g., the intake questionnaire 134). The health engine 114 may also receive a first health goal 110 and/or a second health goal 112 of the user 102 from the user input via the GUI 106 in response to the health questionnaire.

The health questionnaire may include questions, such as, “what time do you awaken in the morning?”; “what time do you exercise?”; “how many times do you eat each day?”; “what time is your first meal?”; “what are the contents of your first meal?”; “what time is your second meal?”; “what are the contents of your second meal?”; “what time is your third meal?”; “what are the contents of your third meal?”; “what time do you go to sleep?”; “how well do you sleep?”; “do you have any past injuries or surgeries?”; “do you have any current or chronic musculoskeletal pain or ailments?”; “do you work out or exercise?”; “how often do you work out or exercise?”; “do you enjoy working out?”; “have you attempted to meet any health or fitness goals before?”; “have you met any health or fitness goals before?”; “are you training to improve in a specific sport?”, etc. It should be appreciated that the listed questions are for illustrative purposes only and the questions are not limited to those explicitly listed herein. Moreover, if the user 102 is training to improve in a specific sport, such as body building, cycling, running, baseball, basketball, tennis, football, etc., the health questionnaire may additionally include the following questions, “have you ever experienced a fracture?”; “do you have any chronic tissue injuries?”; “do you have any joint pain?”, etc.

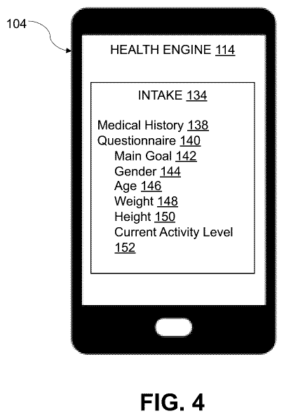

More specifically, as shown in FIG. 4, questions 140 of the intake questionnaire 134 may be based on: a medical history 138 of the user 102, health/wellness goals 142 the user 102 wishes to accomplish (e.g., run a half-marathon), a gender 144 of the user 102, an age 146 of the user 102, a weight 148 of the user 102, a height 150 of the user 102, a current activity level 152 of the user 102, etc. In some examples, the intake questionnaire 134 is a mosaic questionnaire. In the mosaic questionnaire, a portion of the questions may not be filled out from the beginning by the user 102. In other examples, the questions 140 of the intake questionnaire 134 change/evolve based on the input received from the user 102.

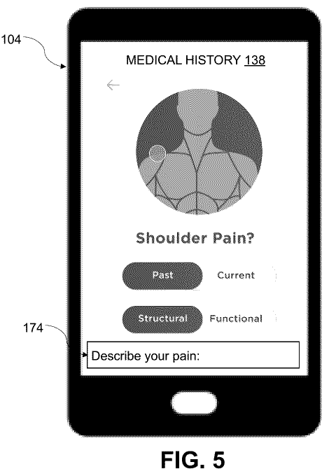

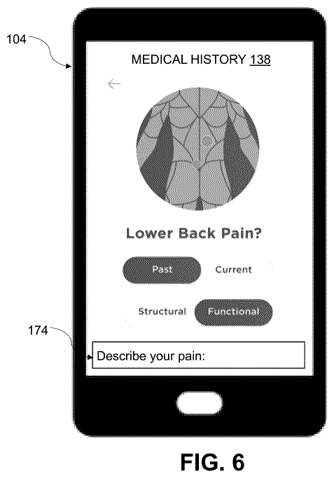

In some examples, the user 102 may indicate or input into the health engine 114 that the user) 102 has or has had some sort of pain (such as shoulder pain, lower back pain, leg pain, etc.). In response, the health engine 114 may display a graphic of such pain, as shown in FIG. 5 and FIG. 6. Moreover, the health engine may prompt the user 102 to select whether such pain is past or current, whether such pain is structural or functional, etc. Moreover, the health engine 114 may prompt the user 102 to describe the pain in a text entry box 174. Such textual description is subsequently analyzed by the health engine 114.

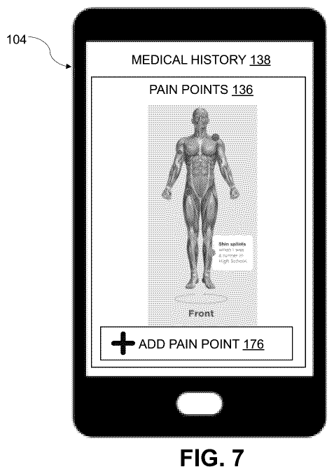

In other examples, and as shown in FIG. 7, the health engine 114 may prompt the user 102 to select pain points 136 on a graphical representation of a human body. The user 102 may also add pain points 176 to the graphical representation of the human body. All of the gathered health data 108 may be used/analyzed by the health engine 114.

The first health goal 110 of the user 102 may be configured for completion during a first time period and the second health goal 112 of the user 102 may be configured for completion during a second time period. In some examples, the first time period is a current time period and the second time period is a future time period. In other examples, the second time period is the current time period and the first time period is the future time period. As an illustrative example, the first health goal 110 for completion during the current time period may include losing body fat and the second health goal 112 for completion during the future time period may include running a marathon. Other examples of the first health goal 110 and/or the second health goal 112 may include: losing weight, improving body composition or tone, building muscle, getting stronger or more powerful, sport specific goals, relieving pain, etc.

In examples, the health data 108 described may include medical data, genetic data, nutritional data, fitness data, and/or environmental data, among other data not explicitly listed herein. In some examples, the health data may additionally include stories of the user that encompass the medical data, the genetic data, subjective measures (e.g., self-reported by the user), the nutritional data, the fitness data, and/or the environmental data. In some examples, the medical data may prove to be important, as it may indicate risk factors for the user 102 for chronic diseases, such as hypertension, hypercholesterolemia, coronary artery disease, cancer, and/or diabetes, etc. Examples of the medical data may include: a known health problem of the user 102, a prior health problem of the user 102, a current health problem of the user 102, a health problem of a family member associated with the user 102, and/or a physiological or biochemical measurement of the user 102, among other medical data not explicitly listed herein. The physiological or biochemical measurement of the user 102 may include: a heart rate measurement, a resting metabolic rate (RMR) measurement, an oxygen consumption (VO2) level measurement, a weight measurement, a body fat measurement, a visceral fat measurement, a muscle mass measurement, a measurement of body water of the user 102, a body mass index (BMI) measurement, a bone mass measurement, and/or a blood glucose level measurement, among other measurements not explicitly listed herein.

In other examples, the genetic data may include genomic information associated with the user 102. In some examples, the genomic information may be targeted and may be specifically related to genetic correlations with diseases. In other examples, the nutritional data may include information such as: types of foods eaten by the user 102, a number of daily calories consumed by the user 102, a quantity of meals consumed daily by the user 102, a quantity of snacks consumed daily by the user 102, a type of snacks consumed daily by the user 102, a type of beverage consumed daily by the user 102, and/or a quantity of beverages consumed daily by the user 102, among other data not explicitly listed herein. It should be appreciated that more questions may be asked than what is visually represented to gain a better understanding of the pain points of the user 102 and how to work with those pain points (e.g., either how to change exercises, avoid exercises, or maybe even suggest contacting a medical professional before returning to exercise).

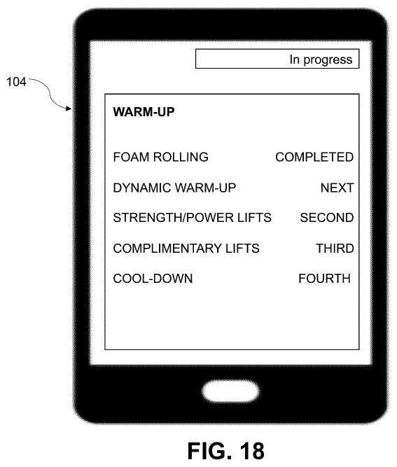

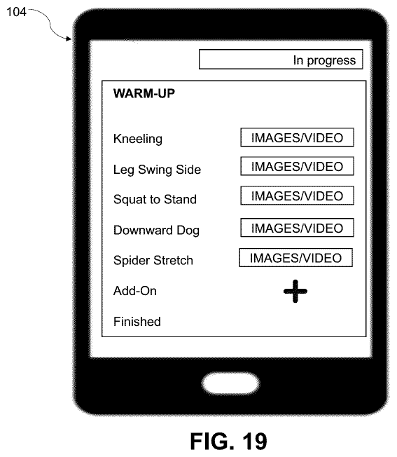

In additional examples, fitness data (as shown in FIG. 18 and FIG. 19) may include: a type of exercise routine engaged in by the user 102, a type of workout engaged in by the user 102, a length of time spent on the exercise routine, a length of time spent on the workout a number of calories burned during the exercise routine, a number of calories burned during the workout, a heart rate achieved during the exercise routine, and/or a heart rate achieved during the workout, among other data not explicitly listed herein. As depicted in FIG. 18 and FIG. 19, the fitness data includes multiple exercises to be completed by the user 102, such as a spider stretch, and a downward dog stretch. As depicted, the user 102 can also view images of the exercises directly on the GUI.

Moreover, examples of the environmental data may include one or more lifestyle choices of the user 102. In examples, the one or more lifestyle choices of the user 102 may include: a sleep habit of the user 102, a type of learner the user 102 is (e.g., a visual learner, an auditory learner, or a heuristic learner), a smoking habit of the user 102, and/or an alcohol intake habit of the user 102, among other data not explicitly listed herein.

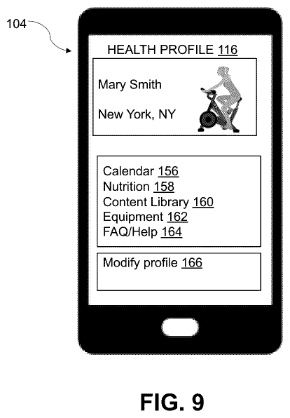

Then, the health engine 114 may create a health profile 116 for the user 102 that includes the health data 108, the first health goal 110, and/or the second health goal 112, among other information. The health profile 116 may also include information about the user, such as a name, an address, a photograph, a graphic, a bitmoji, etc. As shown in FIG. 9, the health profile 116 may include one or more modules, such as a calendar module 156, a nutrition module 158, a content library 160, an equipment module 162, and/or a frequently asked questions (FAQ)/help module 164, among others not explicitly listed herein.

The user 102 may input exercises performed by the user 102 into the calendar module 156. The calendar module 156 may distinguish between different types of exercises by using unique colors, text, font, etc. for each exercise. The calendar module 156 may also be used to track food items or meals eaten by the user 102. The content library 160 may include user-selected infographics, videos, images, books, articles, etc. focused around health/wellness.

The user 102 may also engage in actions to modify 166 the health profile 116, which may include adding content to the health profile 116, deleting content from the health profile 116, and/or cancelling or deleting the health profile 116. The user 102 may also engage in actions to upload the users 102 own picture/photograph or modify an existing picture/photograph.

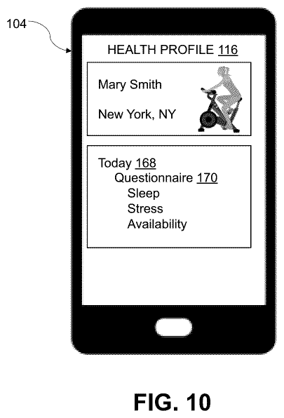

As shown in FIG. 10, the health profile 116 may include a today module 168. The today module 168 may include a questionnaire 170 regarding how the user 102 is feeling on a given day. The questionnaire 170 may pose questions to the user 102, such as an amount of sleep the user 102 got the previous night, a stress level of the user 102, an availability of the user 102, a current energy level of the user, etc. In response to each of the questions of the questionnaire 170, the user 102 may select a specific number (e.g., between 1-20), a quantity of an object (e.g., 4/5 stars), etc.

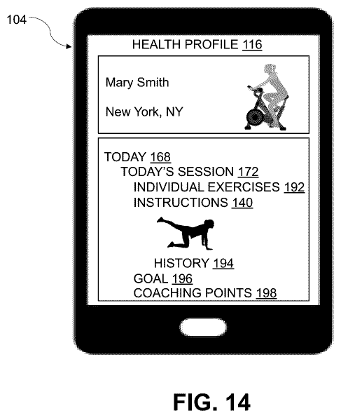

As shown in FIG. 14, the today module 168 may also include information regarding: today's workout session 172, individual exercises 192 within today's workout session 172, instructions on how to perform each of the individual exercises 192, a history 194 regarding the user 102 performing each of the individual exercises 192 and/or the workout session 172, current fitness goals 196 of the user 102, and/or coaching points 198 for the user 102. The user 102 also has the ability to “scramble” or switch up today's workout session 172 or each of the individual exercises 192 within today's workout session 172 based on the users preferences.

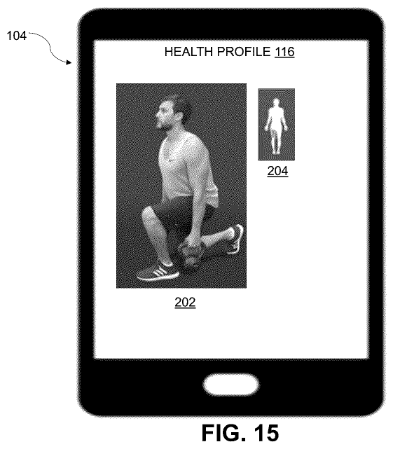

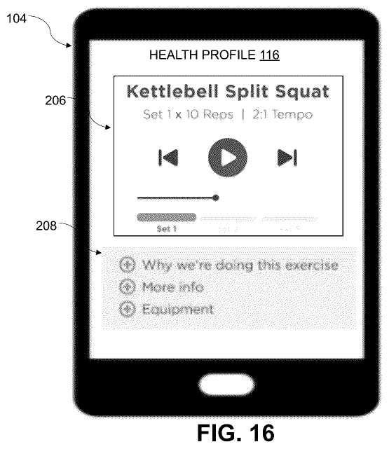

The instructions on how to perform each of the individual exercises 192 within today's workout session 172 may be textual, audio, or graphical (e.g., an image or video). FIG. 15 shows a graphical representation 202 of an individual exercise 192 within today's workout session 172. Moreover, FIG. 15 shows a graphical representation 204 of a body part that each of the individual exercises 192 targets. The instructions on how to perform each of the individual exercises 192 may also include additional information 208 of FIG. 16 regarding why the user 102 is performing this exercise, more information about the exercise, and equipment needed to perform the given exercise.

In some examples, the instructions on how to perform each individual exercises 192 may include a trainer performing such exercise in real-time. Moreover, the coaching points 198 of FIG. 14 may include textual, audio, or graphical (e.g., an image or video) instructions to the user 102 on how to perform each of the individual exercises 192 within today's workout session 172 more accurately.

Moreover, in some implementations, a heatmap component (not shown) may be incorporated into this invention. At any given point while the user 102 is engaging with a given exercise, the heatmap component (executed by the health engine 114) pictorially depicts which muscles should be working. The muscles that should be working may be shown in red, whereas the muscles that should not be working may be shown in blue. Such colors are provided for illustrative purposes only and other colors are contemplated. Such heatmap component may also be affected by the intake questionnaire 134. For example, if the user 102 specifies in the intake questionnaire 134 that the user 102 is short-limbed and long-torsoed, the heatmap component may differ than if the user 102 specifies in the intake questionnaire 134 that the user 102 is short torsoed and long-limbed.

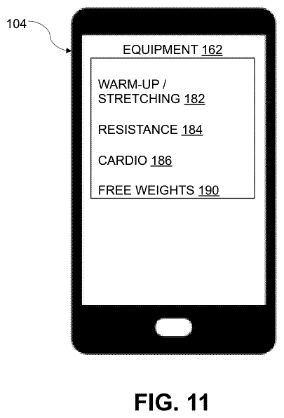

As shown in FIG. 11, the equipment module 162 of the health profile 116 may include categories, such as: warm-up/stretching equipment 182, resistance equipment 184, cardio equipment 186, and/or free weights equipment 190, among others not explicitly listed herein. Each of these categories may include common equipment used for the given category and a list of suggested equipment for purchase 188. The suggested equipment for purchase 188 may include graphics of the suggested equipment, website links to places the user 102 can purchase the suggested equipment, prices of the suggested equipment, physical locations/stores where the user 102 may purchase the suggested equipment, etc. The user 102 may also filter the suggested equipment for purchase 188 based on the users 102 fitness goals, a budget of the user 102, space the user 102 has to dedicate to fitness equipment, etc.

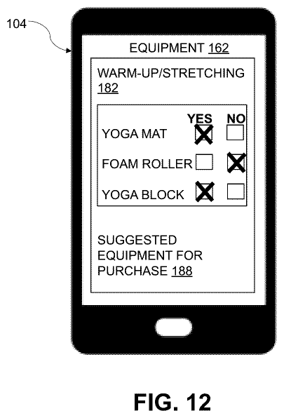

As an illustrative example, the warm-up/stretching equipment 182 category is depicted in more detail in FIG. 12. For example, the warm-up/stretching equipment 182 may include: a yoga mat, a foam roller, and/or a yoga block, among other pieces of equipment not explicitly listed herein. The user 102 may then select which pieces of equipment the user 102 has and/or may browse and/or purchase any of the suggested equipment 188.

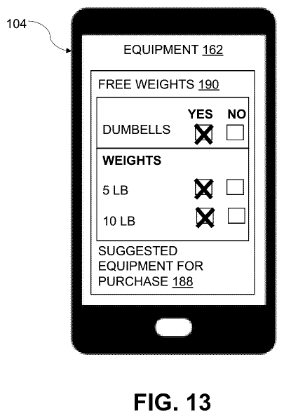

As another illustrative example, the free weights category 190 is depicted in more detail in FIG. 13. As an example, the free weights category 190 may include dumbbells of varying weights (e.g., 2 lbs., 5 lbs., 10 lbs., 20 lbs., etc.). The user 102 may then select which pieces of equipment the user 102 has and/or may browse and/or purchase any of the suggested equipment 188.

Next, the health engine 114 may implement or incorporate the health data 108 in if-then scenarios 120 to determine a first wellness action 122 (e.g., a “need”) for the user 102 to complete during the first time period to achieve the first health goal 110 (e.g., a “want” of the user 102) and a second wellness action 124 (e.g., a “need”) for the user 102 to complete during the second time period to achieve the second health goal 112 (e.g., a “want” of the user 102). As an illustrative example, the user 102 may have the first health goal 110 (e.g., the “want”) of toning the user's overall body and the first wellness action 122 (e.g., the “need”) may be the user 102 engaging in high intensity interval training exercise three to four times a week. If the user 102 does not focus heavily on engaging in the high intensity interval training exercise three to four times a week, as suggested, to meet the first health goal 110, the health engine 114 may not face liability from this user-driven decision.

In some examples, the health engine 114 may use an algorithm 118 during the if-then scenarios 120 to assess all of the health data 108 (or factors) contributing to the health of the user 102 to help the user 102 form healthy habits gradually and intentionally until they are automated, internalized, and intuitive to the user 102. In some examples, the algorithm 118 may include an artificial intelligence (AI) algorithm, a deep learning algorithm, a decision-making algorithm, or an AI computer vision algorithm. However, the algorithm 118 is not limited to these examples explicitly listed herein.

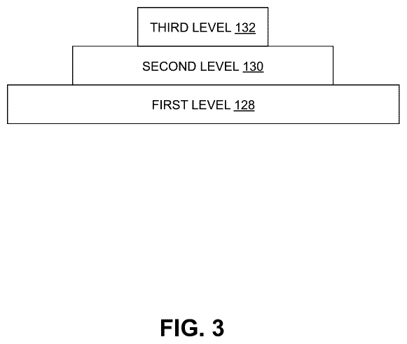

As shown in FIG. 3, the algorithm 118 described herein functions to help the user 102 develop skills that have a logical order and are meant to be built on top of each other to form habits, beginning with more general, foundational elements (e.g., learning the basics of a workout and/or learning basic nutritional principles) (e.g., a first level 128) and moving to finer points (e.g., a second level 130 and a third level 132).

As a first illustrative example, assume that the first health goal 110 for completion during the current time period may include losing body fat. Utilizing the if-then scenarios 120, if the user 102 has a family history of heart disease, then the first wellness action 122 for the user 102 to complete during the first time period to achieve the first health goal 110 of losing body fat may include eating a well-balanced diet. Utilizing the if-then scenarios 120, if the user 102 rarely or never exercises and has no pre-existing injuries, then the first wellness action 122 for the user 102 to complete during the first time period to achieve the first health goal 110 of losing body fat may include eating a well-balanced diet and/or engaging in multiple high intensity interval training sessions. Utilizing the if-then scenarios 120, if the user 102 rarely or never exercises, has no pre-existing injuries, and has met fitness or health goals prior or doesn't easily get discouraged from trying new things, then the first wellness action 122 for the user 102 to complete during the first time period to achieve the first health goal 110 of losing body fat may include eating a well-balanced diet and/or engaging in exercises new to the user 102. Utilizing the if-then scenarios 120, if the user 102 rarely or never exercises, has no pre-existing injuries, and has not met fitness or health goals prior or easily get discouraged trying new things, then the first wellness action 122 for the user 102 to complete during the first time period to achieve the first health goal 110 of losing body fat may include eating a well-balanced diet and/or engaging in exercises known or common to the user 102.

As another illustrative example, assume that the second health goal 112 for completion during the future time period may include running a marathon. Utilizing the if-then scenarios 120, if the user 102 has the family history of heart disease, then the second wellness action 124 for the user 102 to complete during the second time period to achieve the second health goal 112 of running a marathon may include a workout plan whereby the user 102 continually increases the amount of miles he/she runs on a weekly basis. Utilizing the if-then scenarios 120, if the user 102 rarely or never exercises and has no pre-existing injuries, then the second wellness action 124 for the user 102 to complete during the second time period to achieve the second health goal 112 of running a marathon may include eating a well-balanced diet and/or engaging in weekly running sessions.

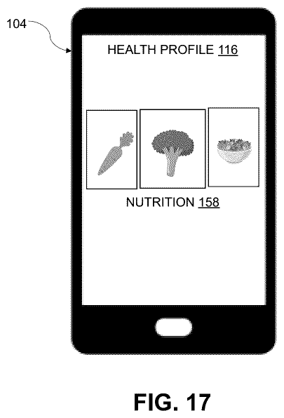

As a further illustrative example, assume that the first health goal 110 for completion during the current time period may include toning one or more muscles of a body of the user 102 and the second health goal 112 for completion during the future time period may include competing in an iron man competition. Part of the health data 108 of the user 102 may include nutritional data (e.g., the user 102 consumes over 2,000 calories daily, with a majority of the calories being present in salty or fatty foods). Furthermore, the user 102 may add or delete food items and/or meals via the nutrition module 158 of FIG. 17.

Utilizing the if-then scenarios 120, if the user 102 is currently consuming over 2,000 calories daily, then the first wellness action 122 for the user 102 to complete during the first time period to achieve the first health goal 110 of toning one or more muscles of the body of the user 102 may include reducing the users 102 caloric intake and replacing some of the salty or fatty foods with high protein food options. Utilizing the if-then scenarios 120, if the user 102 is currently consuming over 2,000 calories daily, then the second wellness action 124 for the user 102 to complete during the second time period to achieve the second health goal 112 of competing in an iron man competition may include reducing the intake of salty or fatty foods and/or eating a paleo or vegan diet. It should be appreciated that the examples provided herein are non-exhaustive and used for illustrative purposes only.

Then, the health engine 114 may create a health and wellness program 126 in the health profile 116 for the user 102 based on the first wellness action 122 and the second wellness action 124. It should be appreciated that the health and wellness program 126 may be displayed to the user 102 via the GUI 106. Moreover, based on the learning style of the user 102, the health and wellness program 126 may be displayed differently to different users. For example, if the user 102 is identified as a visual learner, the health and wellness program 126 may be displayed in numerous charts or graphs. If the user 102 is identified as an auditory learner, the health and wellness program 126 may be displayed via one or more audio or video files to the user 102.

The health and wellness program 126 may include personalized workouts for the user 102, daily or weekly actions for the user 102, nutritional recommendations for the user 102, equipment recommendations for the user 102, supplement recommendations for the user 102, and/or personalized education for the user 102 to help the user 102 meet the first health goal 110 and/or the second health goal 112. The health and wellness program 126 may be presented to the user 102 via one or more of the modules discussed herein. Moreover, the health and wellness program 126 may also include a list of skills acquired by the user 102 over a time period to encourage the user to gain healthy habits.

In some examples, the health engine 114 may receive user activity information pertaining to the user's participation in the health and wellness program 126 (e.g., via user input) and/or may track metrics of user participation in the health and wellness program 126. In response, the health engine 114 may update the health profile 116 based on the user's participation in the health and wellness program 126.

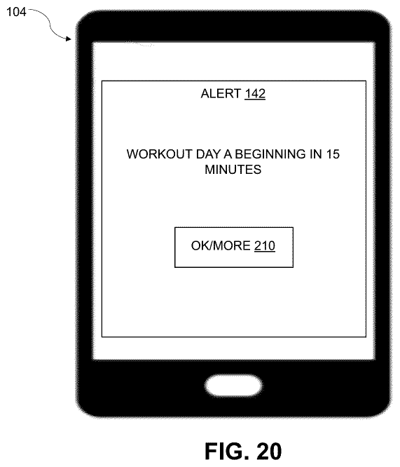

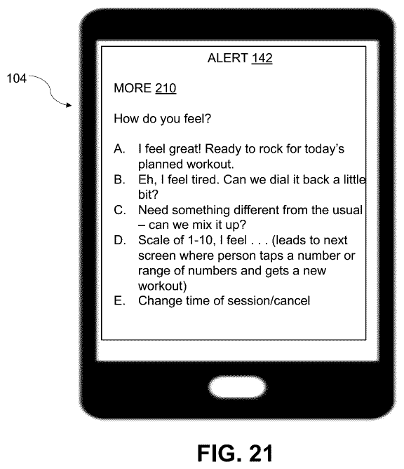

The health engine 114 may also transmit one or more notifications, messages (as depicted in FIG. 21), and/or alerts (as depicted in FIG. 20) to the GUI for display to the user based on the user's participation. The notification may include instructions on how to perform an exercise. As depicted in FIG. 20, the alert may notify or remind the user 102 that a workout or exercise routine should begin in a few minutes. As depicted in FIG. 21, the message may inquire how the user 102 is feeling before or after a particular exercise.

In some examples, the one or more notifications, messages, and/or alerts may include ongoing and personalized education to inform the user 102 on how the user 102 can increase or improve upon his/her performance in the health and wellness program 126. Such ongoing and personalized education may include text, graphics, videos, media, etc. In some examples, the one or more notifications, messages, and/or alerts may include recommendations and/or feedback data to educate, assimilate, and automate the health and wellness program 126 for the user 102. Specifically, the education may include creating user awareness and providing self-directed education based on the user-specific health goals. The assimilation may include turning the education into actionable items (e.g., the first wellness action 122 and/or the second wellness action 124) for the user 102. The automation may include turning the actionable items into habits of the user 102 to create a healthier individual.

In other examples, the one or more notifications, messages, and/or alerts may be generated by a healthcare provider supervising the user's participation in the health and wellness program 126. The healthcare provider may be a nurse, a doctor, a nutritionist, a physical trainer, a fitness coach, etc. In additional examples, the health engine 114 may also provide points and/or rewards to the user 102 based on a level of the user's participation in the health and wellness program 126. In further examples, the health engine 114 may also provide points and rewards to the user 102 to incentivize the user 102 to increase his/her participation in the health and wellness program 126. As has been shown, the health engine 114 implements the method for the real-time fitness tracking and scheduling, which includes providing both a macro-analysis and a micro-analysis of health data 108 of the user 102, assessing the health data 108 in the if-then scenarios 120 to determine user-specific wellness actions for the user 102 to complete to achieve user-specific health goals, and generating a health and wellness program 126 based on these user-specific wellness actions to achieve the user-specific health goals.

It should be appreciated that the health engine 114 may track all data related to the health and fitness of the user 102 continuously and for the long-term. For example, the health engine 114 may track the health data 108 of the user 102, the first health goal 110 of the user 102, the second health goal 112 of the user 102, the user's participation towards execution of the first wellness action 122, the user's participation towards execution of the second wellness action 124, and the user's level of participation in the health and wellness program 126. The tracking by the health engine 114 of such data related to the health and fitness of the user 102 may provide consistent and real-time feedback on the progress of the education process (e.g., the creation of user awareness and the generation of self-directed education based on the user-specific health goals), the assimilation process (e.g., the transformation of the education into actionable items, such as the first wellness action 122 and/or the second wellness action 124) for the user 102, and the automation process (e.g., the transformation of the actionable items into habits of the user 102 to create a healthier individual).

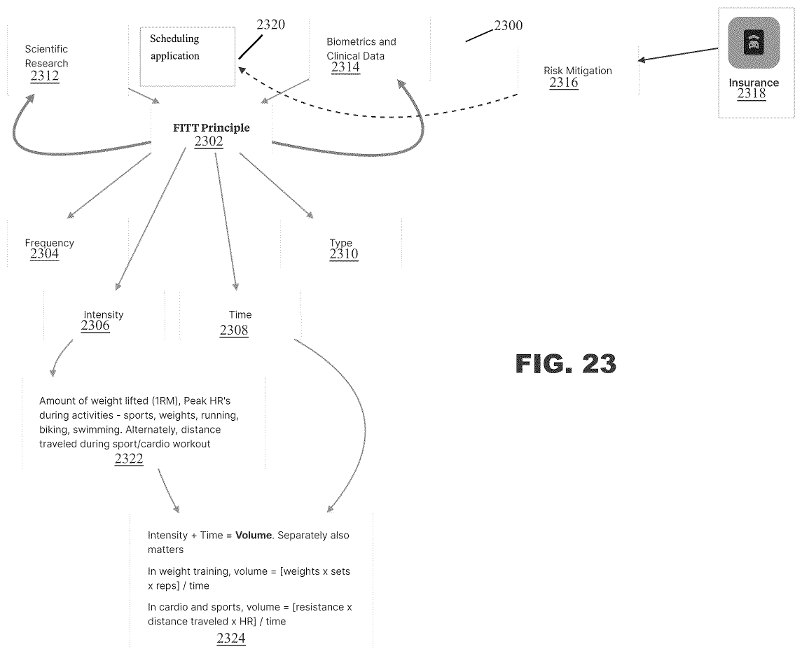

FIG. 23 is a diagram illustrating an example process 2300 for the real-time fitness tracking and scheduling application 2320 implementing the health engine to receive and retain intake, generate assessment forms and perform assessments, receive check-in data and feedback data, generate and communicate SMART goals, plan, and timeline data, promote education, and connect the user to resources. The frequency, intensity, time, and type (FITT) principle 2302, when applied to a fitness tracking app, entails a systematic approach to structuring workout routines. It involves four key components: frequency 2304, intensity 2306, time 2308, and type 2310. Frequency refers to how often workouts are performed, gradually increasing as fitness improves. Intensity pertains to the level of exertion during exercises, which should also progress over time. Time denotes the duration of each workout session, with a gradual increase as endurance builds. Lastly, Type encompasses the variety of exercises incorporated into the regimen, ensuring a well-rounded fitness routine that targets different muscle groups. By adhering to the FITT principle, users can effectively track and optimize their fitness progress through the app, gradually advancing towards higher levels of fitness and performance.

At block 2322, intensity in exercise can be quantified through multiple parameters, including the amount of weight lifted relative to an individual's one-repetition maximum (1RM), peak heart rates attained during various activities such as weightlifting, running, biking, swimming, or engaging in sports, and the distance covered during cardiovascular or sport-specific workouts. These metrics provide objective measures of the physiological demands placed on the body during exercise, reflecting the level of effort expended and the metabolic stress experienced. By accurately assessing intensity through these diverse indicators, athletes, coaches, and researchers can tailor training programs, monitor performance, and optimize training adaptations to achieve specific fitness goals and enhance athletic performance.

At block 2324, Intensity+Time=Volume. In weight training, volume=[weights×sets×reps]/time. In cardio and sports, volume=[resistance×distance traveled×HR]/time. The concept of volume is a metric encompassing both intensity and time. Volume represents the total amount of work performed during a given exercise session and serves as a fundamental determinant of training efficacy and adaptation. While intensity and time are distinct factors, their interplay influences the overall volume and, consequently, the training stimulus. In weight training, volume calculation involves multiplying the weight lifted by the number of sets and repetitions, then dividing by the time taken to complete the workout. This formula quantifies the total workload imposed on the muscles, accounting for both the resistance applied and the duration of the lifting session. By manipulating these variables, athletes and fitness enthusiasts can tailor their training volume to achieve specific strength and hypertrophy goals.

Conversely, in cardiovascular and sports activities, volume calculation considers parameters such as resistance (e.g., incline or terrain), distance traveled, heart rate response, and time duration. The formula for cardiovascular and sports volume incorporates these variables, emphasizing the intensity of the activity, the distance covered, and the duration of the exercise session. This comprehensive approach accounts for the physiological demands imposed on the cardiovascular system and musculature during aerobic and anaerobic efforts. Ultimately, understanding the nuanced relationship between intensity, time, and volume is paramount for designing effective training programs across various modalities. By manipulating these components, athletes and coaches can optimize training adaptations, enhance performance, and mitigate the risk of overtraining or injury.

Scientific research 2312 plays a role in informing and supporting the methodologies used for recommendations. By meticulously analyzing studies, scientific data is extracted to enhance the app's efficacy in guiding users toward optimal fitness outcomes. This involves leveraging existing research-backed methods to measure progress across various health parameters including, but not limited to, biometrics and clinical data 2314, and/or exercise metrics such as weight lifted, reps performed, exercise duration, and heart rate. Moreover, scientific research 2312 facilitates the identification and implementation of validated assessments to monitor users' progress accurately. For instance, assessments such as the Romberg's balance test or the timed up-and-go test can be utilized to evaluate lower-body strength, balance, and fall risk, particularly in aging individuals. The Romberg's balance test assesses the individual's ability to maintain balance while standing still with eyes closed, which challenges proprioception and vestibular function. On the other hand, the timed up-and-go test evaluates mobility and agility by timing how long it takes for the individual to stand up from a chair, walk a short distance, turn around, return to the chair, and sit back down.

Additionally, advanced techniques such as estimating VO2max from wearable heart rate data enable personalized insights into cardiovascular fitness levels. Furthermore, scientific findings serve as a foundation for assessments and risk mitigation 2316, particularly in scenarios involving insurance 2318 such as, insurers and/or payers. By correlating fitness metrics with mortality rates, derived from reputable sources like the New England Journal of Medicine, the app can provide users and insurers with valuable insights into the potential health benefits associated with improving fitness levels. Overall, the fitness app integrates client-entered data, wearable data, and insights gleaned from scientific research to credit individuals for their health efforts accurately. Through this multidimensional approach, users receive tailored recommendations and incentives based on objective evidence, enhancing their motivation and accountability towards achieving their fitness goals.

In some embodiments, within the “options/settings” section of the app, advanced users will have access to sliders, a feature allowing them to manually adjust specific aspects of their experience. These sliders offer a level of customization beyond the automated recommendations generated by the software and AI, catering to the nuanced preferences and goals of experienced individuals. By fine-tuning parameters such as exercise intensity, duration, frequency, or targeted muscle groups, users can exert greater control over their training regimen. This manual adjustment capability empowers advanced clients to tailor their experience according to their evolving needs, preferences, and performance objectives, ultimately enhancing the efficacy and personalization of their fitness journey.

In some embodiments, the intake process of the fitness app involves systematically gathering user data to establish a comprehensive fitness profile. This includes a thorough health history, encompassing past and present medical conditions, medications, allergies, surgeries, and familial medical history. Additionally, users are required to complete a liability waiver, acknowledging and assuming the inherent risks associated with physical activity, absolving the app and its developers of liability. The app also collects detailed exercise history, including frequency, duration, intensity, types of activities, and fitness goals. Moreover, users provide information about available exercise equipment, such as gym equipment, home workout tools, outdoor spaces, or specialized gear, to inform the app's exercise programming. Through the comprehensive collection and analysis of these intake parameters at a highly technical level, the fitness app constructs a detailed user profile. This profile serves as the foundation for delivering personalized recommendations, assessing potential risks, and optimizing fitness programs to provide an individualized and effective fitness experience for each user.

In some embodiments, the fitness app assessment incorporates various analyses to comprehensively evaluate user fitness. This includes movement and flexibility analysis, assessing range of motion and joint mobility. Strength assessment is conducted to gauge muscular capabilities. Cardiovascular endurance evaluation occurs over a longer-term timeline, with algorithmic prioritization based on needs analyses and user preferences. These assessments collectively provide a detailed understanding of the user's fitness profile, enabling the app to tailor recommendations and track progress effectively. For example, the algorithm within the fitness app implements personalizing the user experience and optimizing fitness outcomes. It operates through a multifaceted approach that incorporates data analysis, machine learning, and user feedback to dynamically adapt and refine its recommendations.

In some embodiments, within the “options/settings” section of the app, advanced users will have access to sliders, a feature allowing them to manually adjust specific aspects of their experience. These sliders offer a level of customization beyond the automated recommendations generated by the software and AI, catering to the nuanced preferences and goals of experienced individuals. By fine-tuning parameters such as exercise intensity, duration, frequency, or targeted muscle groups, users can exert greater control over their training regimen. This manual adjustment capability empowers advanced clients to tailor their experience according to their evolving needs, preferences, and performance objectives, ultimately enhancing the efficacy and personalization of their fitness journey.

In some embodiments, the intake process of the fitness app involves systematically gathering user data to establish a comprehensive fitness profile. This includes a thorough health history, encompassing past and present medical conditions, medications, allergies, surgeries, and familial medical history. Additionally, users are required to complete a liability waiver, acknowledging and assuming the inherent risks associated with physical activity, absolving the app and its developers of liability. The app also collects detailed exercise history, including frequency, duration, intensity, types of activities, and fitness goals. Moreover, users provide information about available exercise equipment, such as gym equipment, home workout tools, outdoor spaces, or specialized gear, to inform the app's exercise programming. Through the comprehensive collection and analysis of these intake parameters at a highly technical level, the fitness app constructs a detailed user profile. This profile serves as the foundation for delivering personalized recommendations, assessing potential risks, and optimizing fitness programs to provide an individualized and effective fitness experience for each user.

In some embodiments, the fitness app assessment incorporates various analyses to comprehensively evaluate user fitness. This includes movement and flexibility analysis, assessing range of motion and joint mobility. Strength assessment is conducted to gauge muscular capabilities. Cardiovascular endurance evaluation occurs over a longer-term timeline, with algorithmic prioritization based on needs analyses and user preferences. These assessments collectively provide a detailed understanding of the user's fitness profile, enabling the app to tailor recommendations and track progress effectively. For example, the algorithm within the fitness app implements personalizing the user experience and optimizing fitness outcomes. It operates through a multifaceted approach that incorporates data analysis, machine learning, and user feedback to dynamically adapt and refine its recommendations.

These examples are illustrative and do not represent the full range of the platform's capabilities. The platform is designed to offer flexibility and is not limited to these specific features or assessments. Whether it's advanced users adjusting sliders for personalized control, detailed intake processes gathering health and fitness data, or comprehensive fitness assessments involving movement, strength, and cardiovascular evaluations, the platform is capable of adapting to a variety of user needs. It supports a broad spectrum of features that can be tailored to meet individual preferences and evolving goals, ensuring a fully customizable and responsive experience.

One key aspect of the algorithm is its ability to analyze user data collected during the intake process and throughout their interaction with the app. This includes inputs such as health history, exercise habits, available equipment, movement patterns, and performance metrics. By processing this data, the algorithm generates insights into the user's current fitness status, identifying strengths, weaknesses, and areas for improvement. Moreover, the algorithm leverages needs analysis techniques to prioritize fitness goals and interventions based on the user's profile and objectives. This involves assessing the user's individual needs, preferences, and constraints, then generating tailored recommendations to address them effectively. For example, if a user has a history of lower back pain, the algorithm may prioritize flexibility and core strength exercises to mitigate the risk of injury. Additionally, the algorithm employs machine learning algorithms to continuously refine its recommendations over time. By analyzing user interactions, engagement patterns, and outcomes, the algorithm learns to adapt its suggestions to better align with each user's preferences and evolving needs. This adaptive learning process enhances the app's ability to deliver personalized and effective guidance, fostering long-term engagement and success.

Furthermore, user feedback mechanisms play a role in algorithm refinement. The app solicits input from users regarding the effectiveness of recommendations, perceived difficulty levels, satisfaction with progress, and any barriers encountered. This feedback loop enables the algorithm to iteratively improve its decision-making processes, optimizing the user experience and enhancing fitness outcomes. The algorithm within the fitness app operates as a dynamic and intelligent system that continuously analyzes data, learns from user interactions, and adapts its recommendations to facilitate personalized and effective fitness programming. By leveraging advanced computational techniques and user feedback, the algorithm empowers individuals to achieve their fitness goals more efficiently and sustainably.

In other embodiments, the fitness app's assessment platform can integrate third-party external or licensed technologies, such as advanced cameras, specialized software, or a combination of both. These technologies enable the creation of a highly personalized movement screen and facilitate ongoing feedback to users. Through the utilization of tools, the app can conduct intricate movement analyses with precision and accuracy, capturing detailed data on users' biomechanics, postural alignment, and movement patterns. This information allows for a comprehensive evaluation of the user's movement quality and flexibility, providing valuable insights into areas for improvement and potential injury risk factors. Additionally, the incorporation of real-time feedback mechanisms enables users to receive immediate guidance and corrections during exercises, enhancing the effectiveness and safety of their workouts. By leveraging these advanced technologies within the assessment platform, the fitness app can deliver a more personalized and interactive user experience, promoting optimal movement mechanics and overall fitness outcomes.

Within the fitness platform, users can access comprehensive assessments for posture and stability within the assessment section. This feature encompasses multiple modalities to ensure thorough evaluation and personalized feedback. Users have the option to self-assess and input data or answers based on their observations, providing valuable insights into their own movement patterns and postural alignment. Additionally, the platform offers technology-based assessments, leveraging cameras for imaging or video recordings while users perform specific movements. This allows for detailed analysis of posture and stability, capturing subtle nuances that may not be apparent through self-assessment alone. Furthermore, users have the flexibility to engage with professional assessments, either in-person or virtually, conducted by certified trainers or healthcare professionals. These assessments provide expert guidance and feedback, enhancing the accuracy and depth of the evaluation. Users can choose the assessment method that best suits their preferences and needs, ensuring a tailored and comprehensive experience. To support ongoing progress and optimization, the platform offers re-assessments at intervals determined by the user. Additionally, the software may suggest re-assessments based on user progress, changes in fitness goals, or predefined intervals. This proactive approach to re-assessment ensures that users have regular opportunities to track their progress, identify areas for improvement, and adjust their fitness plans accordingly.

The details provided here are just examples and do not encompass the full range of features the platform can offer. The platform is designed to be versatile and is not restricted to the specific assessments or technologies mentioned. Whether it involves data analysis from the intake process, advanced movement assessments, or feedback mechanisms, the platform can integrate a variety of tools and methodologies to meet diverse user needs. It allows for continuous adaptation and customization, ensuring that users receive personalized fitness guidance tailored to their unique goals and evolving requirements.

In an embodiment of a functional movement analysis protocol focusing on assessments related to shoulder stability and strength, the “Y's” exercise typically involves lifting the arms diagonally upwards and outwards to form a Y shape with the body. It's commonly used to activate and strengthen the muscles of the shoulder girdle, particularly the middle and lower trapezius muscles. The assessment questions associated with “Y's” include determining if the exercise activates the latissimus dorsi muscles (Lats), assessing the strength of the Lats on a scale from 1 to 5, identifying if the upper trapezius muscles are overactive, and assessing for any presence of pain or sticking points during the exercise. The degree of activation of the middle and lower trapezius muscles is also evaluated on a scale from 1 to 5. If the upper trapezius muscles are found to be overactive, further assessment is conducted on a similar scale. These assessments help to identify potential areas of weakness, imbalance, or dysfunction in the shoulder complex, guiding the development of targeted corrective exercises and interventions to improve shoulder stability, strength, and function.

In some examples, “W's” and “L's” are exercises commonly used in functional movement analysis protocols to assess the strength and stability of muscles around the shoulder girdle. In the “W's” exercise, participants lift their arms diagonally upwards and outwards to form a W shape with their bodies, aiming to activate and strengthen the rhomboid muscles. During the assessment, evaluators observe if the exercise activates the rhomboid muscles and assess their strength using a scale from 1 to 5, while also noting any limitations or movement pathologies. Conversely, the “Stability Ball L's” exercise involves performing L-shaped movements while lying face down on a stability ball, targeting the muscles of the shoulder girdle and core for stability. During the assessment of “Stability Ball L's,” evaluators observe for movement pathology in the rhomboid muscles, assess their strength on a scale from 1 to 5, and evaluate the activation and range of motion of the rotator cuff muscles. These assessments are critical for identifying weaknesses, imbalances, or dysfunctions in the shoulder girdle muscles, guiding the development of targeted corrective exercises and interventions to improve shoulder stability, strength, and function.

The assessment measures various parameters related to VO2max, a key indicator of cardiovascular fitness. VO2max is expressed in milliliters per kilogram per minute, providing insight into the body's maximum oxygen uptake capacity during exercise. Additionally, the assessment includes the measurement of power output in watts, representing the amount of work performed by the individual during exercise. Maximum heart rate (HR) in beats per minute (bpm) is also recorded, indicating the highest heart rate achieved during exercise. Furthermore, the assessment outlines specific training heart rate zones based on percentages of maximum heart rate, including zones ranging from 60% to 95% of maximum heart rate. These heart rate zones are crucial for optimizing training intensity and ensuring effective cardiovascular conditioning.

In some embodiments, the assessment involves a series of exercises targeting core stability and overall movement quality. The quadruped section includes exercises like Bird Dog and Fire Hydrants, which challenge core stability and hip mobility. Additionally, the Overhead Squat is assessed for various factors including range of motion (ROM), shoulder and latissimus dorsi (Lats) mobility, trunk stability, hip depth, hamstring strength and mobility, reliance on quadriceps, and stiffness in calves. These assessments provide insights into the individual's movement patterns, identifying areas of strength, weakness, and potential dysfunction. Based on the findings, physical priorities are established, emphasizing the importance of moving well, moving powerfully, and moving intensely. Finally, strength and conditioning recommendations are tailored to address the specific needs identified during the assessment, ensuring targeted and effective programming to improve overall movement quality and physical performance.

In more embodiments, within the fitness platform's intake process, users have the option to refine their fitness goals based on their occupation, allowing for more targeted and personalized programming. Users can input details such as their occupation, industry, and the number of hours spent working each week. Additionally, users can specify the tasks or activities they commonly perform during their workday, indicating the time or percentage spent on various tasks. For example, a construction worker may spend a significant portion of their day lifting heavy objects and engaging in physically demanding tasks, whereas an office worker may spend most of their time seated at a desk. A truck driver, on the other hand, may spend long hours sitting behind the wheel with limited opportunities for physical activity. Based on this information, the fitness platform can tailor programming to address the specific needs and challenges associated with each occupation. For the construction worker, the program may focus on building strength, endurance, and flexibility to support the physical demands of their job. The program for the office worker may prioritize exercises to counteract the effects of prolonged sitting, improve posture, and reduce the risk of musculoskeletal issues. Meanwhile, the program for the truck driver may emphasize exercises to promote mobility, reduce stiffness, and enhance cardiovascular health during long periods of sedentary driving. By incorporating occupation-specific data into the intake process, the fitness platform ensures that users receive programming that is relevant, practical, and aligned with their lifestyle and job requirements.

The assessment process encompasses several key areas to gather comprehensive information about the individual's fitness journey and preferences. The general intake section delves into factors such as available time for exercise, specific goals, level of commitment, and other activities enjoyed, helping to establish priorities and tailor the fitness plan accordingly. Past medical history is thoroughly reviewed to determine if the individual is cleared for exercise and to identify any past or current injuries, medical conditions, or medications that may impact their training. Additionally, the assessment considers the individual's learning style, whether visual, auditory, or heuristic, to optimize the delivery of instruction and feedback. Aesthetic preferences, including body type, Q-angle, and desired look, are also explored to align with the individual's goals and establish priorities, while also considering how these preferences may influence exercise selection and programming. Furthermore, the assessment explores the individual's openness to trying new things, potentially incorporating gamification elements and leveling up strategies to enhance engagement and motivation. Lastly, the assessment addresses the incorporation of external resources such as the “8 Colors of Fitness,” considering licensing and permission issues to ensure ethical and legal use of third-party content in the fitness programming.

In further embodiments, the fitness assessment process is a blend of methodologies, drawing from both external sources and internal expertise. It incorporates elements from the Functional Movement Screening (FMS) provided by a third party, Functional Movement Systems, along with traditional physical therapy assessments. However, the fitness app's approach differs in that the system evaluates these assessments subjectively, leveraging the expertise of exercise physiologists and trainers. In contrast to the numeric scoring commonly used by physical therapists for documentation and insurance purposes, the fitness app prioritizes qualitative analysis, with some quantitative measures potentially included. This enables a more in-depth understanding of users' movement patterns, flexibility, and functional abilities, allowing for more accurate tailoring of exercise prescriptions to meet their specific needs and goals. Through this comprehensive and detailed assessment, the app seeks to offer a personalized and holistic fitness experience for each user.

In some embodiments of implementing the tracking app is to conduct initial assessments of users before engaging with Health and Sports Science (HSS) services. However, in certain scenarios, the app may seek to leverage the superior quality of content from HSS's Fitness Assessment. While the tracking app provides a robust framework for assessing users' fitness levels and needs independently, it acknowledges that HSS's Fitness Assessment may offer additional depth and precision in certain areas. Therefore, in some embodiments, the app may opt to integrate data or insights from HSS's assessment to enrich its own analysis and recommendations. This collaborative approach ensures that users receive the most comprehensive and accurate evaluation possible, drawing from both the app's capabilities and the expertise of HSS. By leveraging the strengths of both platforms, users can benefit from a holistic and tailored fitness experience that addresses their unique needs and goals effectively.

The Fitness platform offers users the functionality to conduct check-ins both before and at the beginning of each session. As users make their way to the gym floor or prepare for their workout, they have the opportunity to communicate how they feel through the app. A virtual trainer then engages with them through targeted questions to gather additional insights. This interactive process allows the app to customize the upcoming session based on the user's most recent intake information and past session data. By leveraging this real-time feedback loop, the app can dynamically adjust the workout plan to accommodate the user's current physical and mental state, ensuring a tailored and optimized exercise experience. This personalized approach enhances user engagement and satisfaction while maximizing the effectiveness of each training session.

Within the Fitness platform, users can provide real-time feedback on their form during exercise sessions. This feature allows trainers to interact with users, either immediately or at the end of a set, to assess their form and technique. To facilitate this process, the platform incorporates advanced technology, such as that provided by Altis, which enables precise movement analysis and feedback delivery. Through this technology, trainers can monitor users' movements in real time, identifying any deviations from proper form and providing corrective feedback as needed. Additionally, users have the opportunity to provide feedback on their own perception of their form, enhancing their awareness and facilitating continuous improvement. By integrating this feedback loop into the training process, the Fitness platform ensures that users receive personalized guidance to optimize their form, minimize injury risk, and increase the effectiveness of their workouts.

Within the fitness platform, users have the capability to provide feedback on the perceived difficulty and enjoyment level of individual exercises. This feedback mechanism is facilitated by trainers who prompt users for their input either in real-time during the exercise or at the conclusion of a set. Currently, users can choose which sets they wish to provide feedback for, granting them flexibility in their engagement with the feedback process. At the end of a set, the trainer presents a question regarding the user's experience of the exercise. The user can then respond by tapping on their preferred answer or swiping left if they found the exercise particularly challenging or unenjoyable. Moreover, users have the option to mark certain exercises off their list if they wish to exclude them from future workouts. For instance, if a user strongly dislikes burpees, they can designate it as an exercise they do not want to include in their routine. This feedback loop allows users to actively contribute to the customization of their workout experience, ensuring that exercises are both challenging and enjoyable. By incorporating user preferences and experiences into the training regimen, the Fitness platform enhances user engagement and satisfaction, ultimately promoting adherence to the program and facilitating progress towards fitness goals.

Within the fitness platform, users have the capability to provide feedback on any discomfort or pain experienced during their workouts. This feedback mechanism may ensure user safety and optimizing their exercise experience. Users are prompted by the trainer to report any discomfort or pain in real-time during the exercise or immediately after completing a set. For example, when a user is performing a squat exercise and starts to feel discomfort in their knees. The trainer would prompt the user to indicate this discomfort through the app. The user can then select the appropriate option to convey the level of discomfort experienced, whether it's mild discomfort, moderate pain, or severe discomfort. Additionally, users may have the option to provide specific details about the location and nature of the discomfort, such as whether it's in the knees, lower back, or elsewhere. By collecting this feedback, trainers can tailor the workout in real-time to address any issues and prevent potential injuries. For instance, if a user reports discomfort in their knees during squats, the trainer may suggest modifying the exercise or substituting it with a different movement that is less taxing on the knees. Overall, the ability to collect feedback on discomfort or pain enhances user safety and ensures that workouts are both effective and enjoyable. By promptly addressing any discomfort reported by users, the Fitness platform fosters a supportive and responsive training environment, promoting long-term adherence and success in achieving fitness goals.

In some embodiments within the fitness platform, users have access to a robust communication feature designed to convey SMART (Specific, Measurable, Achievable, Relevant, Time-bound) goals and personalized workout plans directly to the client. This communication capability is integrated into the user interface, accessible both from the client's homepage and during in-session workouts for convenience and accessibility. For instance, during the initial consultation or assessment, trainers utilize the communication feature to discuss and establish SMART goals with the client. These goals are tailored to the client's individual needs, preferences, and fitness aspirations. The trainer outlines a clear plan, detailing the specific exercises, target metrics, and timelines for achieving each goal. This information is then transmitted directly to the client via the fitness platform, ensuring transparency and alignment between the trainer and the client. Throughout each session, the communication feature remains readily accessible, allowing trainers to provide real-time guidance, encouragement, and adjustments to the workout plan as needed. For example, if the client encounters challenges or barriers during a workout, they can easily communicate with their trainer through the platform to receive immediate support and guidance. Additionally, the communication feature serves as a tool for progress tracking and accountability. Trainers can send regular updates, reminders, and motivational messages to keep clients engaged and on track towards their goals. Clients, in turn, can communicate their progress, concerns, and achievements with their trainer, fostering a collaborative and supportive coaching relationship.

Within the fitness platform, users have access to a robust educational feature designed to provide ongoing learning opportunities tailored to their individual needs and interests. This educational capability is dynamic and responsive, adapting based on user interactions and conversations with trainers. For example, let's say a user expresses interest in learning more about proper nutrition to support their fitness goals. The educational feature would provide curated resources, articles, videos, and interactive modules on topics such as macronutrient ratios, meal planning, and nutrient timing. Trainers may also initiate educational discussions during sessions, offering insights and guidance on relevant topics based on the user's progress and questions. To enhance the educational experience further, the fitness platform establishes third-party portals and partnerships with reputable organizations and experts in various health and wellness domains. These partnerships streamline access to additional educational resources, tools, and services, reducing friction and making it easier for clients to connect with the information they need. For instance, the platform may collaborate with nutritionists, physiotherapists, mental health professionals, or fitness equipment manufacturers to provide users with exclusive access to specialized content, webinars, workshops, and product discounts. By leveraging these partnerships, users can expand their knowledge, skills, and resources beyond what is available within the platform itself. The educational feature within the fitness platform serves as a resource for users seeking to deepen their understanding of fitness, nutrition, and overall wellness. Through curated content and strategic partnerships, users can access educational materials and support services, empowering them to make informed decisions and achieve sustainable lifestyle changes.

Within the fitness platform, users benefit from a connection to the equipment and tools essential for their workouts. This process begins during the initial intake assessment, where users discuss their equipment preferences and needs with their trainer. Whether it's dumbbells, resistance bands, or yoga mats, the trainer helps users identify the equipment necessary to achieve their fitness goals effectively. Throughout their fitness journey, the platform facilitates ongoing discussions and adjustments regarding equipment needs during each session. Trainers communicate with clients to ensure they have access to the right tools for their workouts, whether at home or while traveling. For example, if a client plans to be on the road, the trainer might recommend compact, portable equipment suitable for hotel room workouts. To further streamline the process, the platform provides convenient web links to recommended equipment for purchase. These links direct users to trusted retailers or third-party portals where they can easily browse and purchase the recommended items. By leveraging strategic partnerships with leading fitness equipment providers, the platform ensures that clients have access to high-quality, reliable gear without encountering unnecessary friction. Moreover, the platform's third-party portal and partnerships extend beyond equipment procurement to offer a comprehensive suite of fitness resources. Clients can access additional tools, such as workout apps, nutrition guides, and recovery aids, through these partnerships. This holistic approach to fitness support enables clients to easily connect with the tools they need to enhance their workout experience and achieve their fitness goals efficiently. By integrating equipment recommendations, convenient purchasing options, and strategic partnerships into the fitness platform, users can navigate their fitness journey with confidence, knowing they have access to the resources.

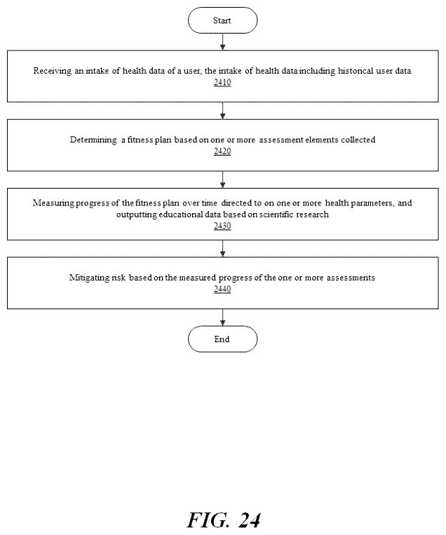

FIG. 24 is a flowchart that describes a method, according to some embodiments of the present disclosure. In some embodiments, at 2410, the method may include receiving an intake of health data of a user, the intake of health data including historical user data. At 2420, the method may include determining a fitness plan based on one or more assessment elements collected. At 2430, the determining may include measuring progress of the fitness plan over time directed to on one or more health parameters, and outputting educational data based on scientific research. At 2440, the determining may include mitigating risk based on the measured progress of the one or more assessments.

In some embodiments, the one or more assessment elements may be aesthetic data. In some embodiments, the aesthetic data may be selected from the group consisting of body type, metabolism type, and Q-angle. In some embodiments, the one or more assessment elements may be basal metabolic rate data. In some embodiments, the basal metabolic rate data may be selected from the group consisting of, calories per day, calories burned, and calories eaten.

In some embodiments, the one or more assessment elements may be functional movement analysis data. In some embodiments, the functional movement analysis data may include one or more of a prone shoulder assessment, quadruped, overhead squat, and physical properties. In some embodiments, the one or more assessment elements are based on functional movement and physical therapy. In some embodiments, the one or more assessment elements may be implemented by a user, artificial intelligence, a third party, or an administrator.

In some embodiments, the one or more health parameters may be received from one or more wireless health devices tracking one or more biometric parameters of the user. In some embodiments, based on a frequency of an activity, an intensity of the activity, a time performing the activity, and a type of activity, implementing an algorithm using an equation, the method may include performing one or more additional steps using the equation Intensity×Time=Volume, to structure a workout routine for the fitness plan. In some embodiments, the measured progress may be determined by a Romberg's balance test or a timed up-and-go test.

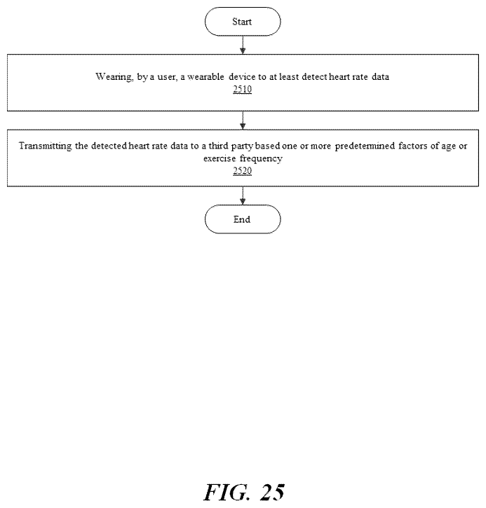

FIG. 25 is a flowchart that further describes the method from FIG. 24, according to some embodiments of the present disclosure. In some embodiments, at 2510, the method may include wearing, by a user, a wearable device to at least detect heart rate data. At 2520, the method may include transmitting the detected heart rate data to a third party based one or more predetermined factors of age or exercise frequency.

### Computer System

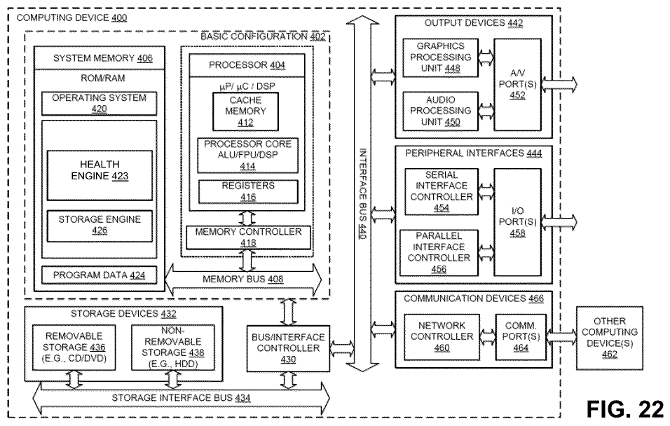

In some embodiments, the present invention may be a computer system, a method, and/or a computing device 104 (of FIG. 1) or 400 (of FIG. 22). For example, the computer system and/or the computing device 400 may be utilized to implement a method for real-time fitness tracking and scheduling.

A basic configuration 402 of a computing device 400 is illustrated in FIG. 22 by those components within the inner dashed line. In the basic configuration 402 of the computing device 400, the computing device 400 includes a processor 404 and a system memory 406. In some examples, the computing device 400 may include one or more processors and the system memory 406. A memory bus 408 is used for communicating between the one or more processors 404 and the system memory 406.

Depending on the desired configuration, the processor 404 may be of any type, including, but not limited to, a microprocessor (μP), a microcontroller (μC), and a digital signal processor (DSP), or any combination thereof. Further, the processor 404 may include one more levels of caching, such as a level cache memory 412, a processor core 414, and registers 416, among other examples. The processor core 414 may include an arithmetic logic unit (ALU), a floating point unit (FPU), and/or a digital signal processing core (DSP Core), or any combination thereof. A memory controller 418 may be used with the processor 404, or, in some implementations, the memory controller 418 may be an internal part of the memory controller 404.

Depending on the desired configuration, the system memory 406 may be of any type, including, but not limited to, volatile memory (such as RAM), and/or non-volatile memory (such as ROM, flash memory, etc.), or any combination thereof. The system memory 406 includes an operating system 420, one or more engines, such as a health engine 423, and program data 424. In some embodiments, the health engine 423 may be a health application, a health software program, a health service, or a health software platform. Moreover, in additional examples, the health engine 423 may comprise the algorithm 118.

The health engine 423 may receive the health data 108 pertaining to the user 102. The health data 108 may include medical data, genetic data, nutritional data, fitness data, and/or environmental data. The health engine 423 may also receive the first health goal 110 of the user 102 for completion during the first time period and the second health goal 112 of the user 102 for completion during the second time period. The health engine 423 may generate the health profile 116 for the user 102 that includes the health data 108, the first health goal 110, and the second health goal 112. Then, the health engine 423 may incorporate or implement, via the algorithm 118, the health data 108 in if-then scenarios 120 to determine the first wellness action 122 for the user 102 during the first time period to achieve the first health goal 110 and the second wellness action 124 for the user 102 during the second time period to achieve the second health goal 112. The health engine 423 may also create the health and wellness program 126 in the health profile 116 for the user 102 based on the first wellness action 122 and the second wellness action 124. Further, the computing device 400 may comprise a storage engine 426, which may be configured to store information, such as the health data 108, the first health goal 110 of the user 102, the second health goal 112 of the user 102, the health profile 116 for the user 102, the first wellness action 122 for the user 102, the second wellness action 124 for the user 102, and/or the health and wellness program 126, among other data not explicitly listed herein.

Moreover, the computing device 400 may have additional features or functionality, and additional interfaces to facilitate communications between the basic configuration 402 and any desired devices and interfaces. For example, a bus/interface controller 430 is used to facilitate communications between the basic configuration 402 and data storage devices 432 via a storage interface bus 434. The data storage devices 432 may be one or more removable storage devices 436, one or more non-removable storage devices 438, or a combination thereof. Examples of the one or more removable storage devices 436 and the one or more non-removable storage devices 438 include magnetic disk devices (such as flexible disk drives and hard-disk drives (HDD)), optical disk drives (such as compact disk (CD) drives or digital versatile disk (DVD) drives), solid state drives (SSD), and tape drives, among others.

In some embodiments, an interface bus 440 facilitates communication from various interface devices (e.g., one or more output devices 442, one or more peripheral interfaces 444, and one or more communication devices 466) to the basic configuration 402 via the bus/interface controller 430. Some of the one or more output devices 442 include a graphics processing unit 448 and an audio processing unit 450, which are configured to communicate to various external devices, such as a display or speakers, via one or more A/V ports 452. The one or more peripheral interfaces 444 may include a serial interface controller 454 or a parallel interface controller 456, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 458. Further, the one or more communication devices 466 may include a network controller 460, which is arranged to facilitate communication with one or more other computing devices 462 over a network communication link via one or more communication ports 464. The one or more other computing devices 462 include servers, the database 106, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 406, the one or more removable storage devices 436, and the one or more non-removable storage devices 438 are examples of the computer-readable storage media. The computer-readable storage media is a tangible device that can retain and store instructions (e.g., program code) for use by an instruction execution device (e.g., the computing device 400). Any such, computer storage media is part of the computing device 400.

The computer readable storage media/medium can be a tangible device that can retain and store instructions for use by an instruction execution device. The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Aspects of the present invention are described herein regarding illustrations and/or block diagrams of methods, computer systems, and computing devices according to embodiments of the invention. It will be understood that each block in the block diagrams, and combinations of the blocks, can be implemented by the computer-readable instructions (e.g., the program code).

The computer-readable instructions are provided to the processor 404 of a general purpose computer, special purpose computer, or other programmable data processing apparatus (e.g., the computing device 400) to produce a machine, such that the instructions, which execute via the processor 404 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored therein comprises an article of manufacture including instructions, which implement aspects of the functions/acts specified in the block diagram blocks.

The computer-readable instructions (e.g., the program code) are also loaded onto a computer (e.g. the computing device 400), another programmable data processing apparatus, or another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks.

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer/computing device, partly on the user's computer/computing device, as a stand-alone software package, partly on the user's computer/computing device and partly on a remote computer/computing device or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to block diagrams of methods, computer systems, and computing devices according to embodiments of the invention. It will be understood that each block and combinations of blocks in the diagrams, can be implemented by the computer readable program instructions.

The block diagrams in the Figures illustrate the architecture, functionality, and operation of possible implementations of computer systems, methods, and computing devices according to various embodiments of the present invention. In this regard, each block in the block diagrams may represent a module, a segment, or a portion of executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block and combinations of blocks can be implemented by special purpose hardware-based systems that perform the specified functions or acts or carry out combinations of special purpose hardware and computer instructions.

Another embodiment of the invention provides a method that performs the process steps on a subscription, advertising, and/or fee basis. That is, a service provider can offer to assist in the method steps for real-time fitness tracking and scheduling. In this case, the service provider can create, maintain, and/or support, etc. a computer infrastructure that performs the process steps for one or more customers. In return, the service provider can receive payment from the customer(s) under a subscription and/or fee agreement, and/or the service provider can receive payment from the sale of advertising content to one or more third parties.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims. Moreover, the particular embodiments disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular embodiments disclosed above may be altered or modified and all such variations are considered within the scope of the disclosed subject matter. Accordingly, the protection sought herein is as set forth in the claims below.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method executed by a health engine of a computing device for real-time fitness tracking and scheduling, the method comprising:
receiving an intake of health data of a user, the intake of health data including historical user data;
determining a fitness plan based on one or more assessment elements collected;
measuring progress of the fitness plan over time directed to on one or more health parameters, and outputting educational data based on scientific research; and
mitigating risk based on the measured progress of the one or more assessments.

2. The method of claim 1, wherein the one or more assessment elements is aesthetic data.

3. The method of claim 2, wherein the aesthetic data is selected from the group consisting of: body type, metabolism type, and Q-angle.

4. The method of claim 1, wherein the one or more assessment elements is basal metabolic rate data.

5. The method of claim 4, wherein the basal metabolic rate data is selected from the group consisting of: calories per day, calories burned, and calories eaten.

6. The method of claim 1, wherein the one or more assessment elements is functional movement analysis data.

7. The method of claim 6, wherein the functional movement analysis data includes one or more of a prone shoulder assessment, quadruped, overhead squat, and physical properties.

8. The method of claim 1, wherein the one or more assessment elements based on functional movement and physical therapy.

9. The method of claim 1, wherein the one or more assessment elements may be implemented by a user, artificial intelligence, a third party, or an administrator.

10. The method of claim 1, wherein the one or more health parameters is received from one or more wireless health devices tracking one or more biometric parameters of the user.

11. The method of claim 1, further comprising:
based on a frequency of an activity, an intensity of the activity, a time performing the activity, and a type of activity, implementing an algorithm using an equation: Intensity+Time=Volume, to structure a workout routine for the fitness plan.

12. The method of claim 1, wherein the measured progress is determined by a Romberg's balance test or a timed up-and-go test.

13. The method of claim 1, further comprising:
wearing, by a user, a wearable device to at least detect heart rate data; and
transmitting the detected heart rate data to a third party based one or more predetermined factors of age or exercise frequency.

14. A computer system comprising one or more processors, one or more memories, and one or more computer-readable hardware storage devices, the one or more computer-readable hardware storage devices containing program code executable by the one or more processors via the one or more memories to implement a method for real-time fitness tracking and scheduling, the method comprising:
receiving an intake of health data of a user, the intake of health data including historical user data;
determining a fitness goal based on one or more assessment elements collected;
measuring progress of the fitness goal over time directed to on one or more health parameters, and outputting educational data based on scientific research; and
mitigating risk based on the measured progress of the one or more assessments.

15. The method of claim 14, wherein the one or more assessment elements is aesthetic data selected from the group consisting of: body type, metabolism type, and Q-angle.

16. The method of claim 14, wherein the one or more assessment elements is basal metabolic rate data selected from the group consisting of: calories per day, calories burned, and calories eaten.

17. The method of claim 14, wherein the one or more assessment elements is functional movement analysis data including one or more of a prone shoulder assessment, quadruped, overhead squat, and physical properties.

18. The method of claim 14, wherein the one or more health parameters is received from one or more wireless health devices tracking one or more biometric parameters of the user.

19. The method of claim 14, further comprising:
based on a frequency of an activity, an intensity of the activity, a time performing the activity, and a type of activity, implementing an algorithm using an equation: Intensity×Time=Volume, to structure a workout routine for the fitness goal.

20. The method of claim 14, wherein the measured progress is determined by a Romberg's balance test or a timed up-and-go test.
